(19) United States
(12) Patent Application Publication
(10) Pub. No.: US 2023/0006573 A1
(43) Pub. Date: Jan. 5, 2023

(54) MEDICAL ULTRASONIC TRIBOELECTRIC GENERATOR STRUCTURE FOR CHARGING BODY IMPLANTABLE DEVICE AND METHOD OF FORMING THE SAME

(71) Applicant: ENERGY MINING Co., Ltd., Suwon-si (KR)

(72) Inventors: Hyun-Moon PARK, Suwon-si (KR); Youngmin CHO, Suwon-si (KR); Jang-Mook JEONG, Suwon-si (KR)

(73) Assignee: ENERGY MINING Co., Ltd., Suwon-si (KR)

(21) Appl. No.: 17/855,052

(22) Filed: Jun. 30, 2022

(30) Foreign Application Priority Data
Jun. 30, 2021 (KR) 10-2021-0085230

(51) Int. Cl. H02N 1/04

(52) U.S. Cl. CPC H02N 1/04; A61N 1/3787

(57) ABSTRACT

The present disclosure relates to a medical ultrasonic triboelectric generator structure for charging a body implantable device and a method of forming the structure. A method of forming a medical ultrasonic triboelectric generator structure for charging a body implantable device includes (a) primarily performing a plasma process on a power generation material on which a polymer material is disposed and performing bonding of the polymer material and a non-conductive material, and (b) secondarily reinforcing the bonding using a physical guide structure including a non-conductive guide structure and a fixing coupling structure.

### CROSS-REFERENCE TO RELATED APPLICATION

This application claims priority to and the benefit of Korean Patent Application No. 10-2021-0085230, filed on Jun. 30, 2021, and Korean Patent Application No. 10-2022-0024681, filed on Feb. 24, 2022, the disclosure of which is incorporated herein by reference in its entirety.

### BACKGROUND

### 1. Field

The present disclosure relates to a technology for performing a surface process on an ultrasonic-based triboelectric generator and arranging a physical guide thereon to perform charging of a body implantable device.

This research was supported by the Accelerator Investment-Driven TIPS(Tech Incubator Program for Startup)(No.S3282292, Power generation and platform technology for charging implantable medical devices). This research was supported by the Bio & Medical Technology Development Program of the National Research Foundation (NRF)& funded by the Korean government (MSIT)(No. 2022M3E5E9016662, Development of Electroceuticals Based on Energy Mining Technology for Obesity Treatment).

### 2. Discussion of Related Art

For application of body implantable charging elements, non-toxicity, harmlessness, and high durability should be ensured.

In the triboelectric charging method for a triboelectric charging generator according to the related art, two materials having different orders of electrification are used on a non-conductive material substrate. According to the related art, it is difficult to attach substances applied to power generation materials to a substrate, and thus a chemical etching method and a plasma processing method have been proposed. The chemical etching method is harmful to the environment and causes coloring. A heat-based oxygen plasma processing method has a problem of low adhesive strength which degrades reliability of a product.

Further, power is generated by contact/separation due to a potential difference between charged objects vibrating at a high speed. When the potential difference between two elements is excessive and an attractive force therebetween is greater than a physical force for the contact/separation, the two elements are adsorbed due to an electrostatic attractive force, and accordingly, power is not generated.

### SUMMARY

The present disclosure proposes a medical ultrasonic triboelectric generator structure for charging a body implantable device, in which a plasma surface process is primarily performed and an adhesion strength is secondarily increased using a physical guide structure so that power generation efficiency of a triboelectric generator (TENG)-based power generator can be improved, bonding of heterogeneous or homogeneous materials can be reliably performed, and a short circuit and inflow of foreign substances can be prevented, and a method of forming the structure.

A method of forming a medical ultrasonic triboelectric generator structure for charging a body implantable device according to the present disclosure includes (a) primarily performing a plasma process on a power generation material on which a polymer material is disposed and performing bonding of the polymer material and a non-conductive material, and (b) secondarily reinforcing the bonding of the polymer material and the non-conductive material using a physical guide structure including a non-conductive guide structure and a fixing coupling structure.

The polymer material may be disposed on a metal material and bonded to the non-conductive material in preset regions at both ends of the metal material, and the non-conductive guide structure may be provided in a “c” shape and is disposed in a shape surrounding partial regions of the polymer material, the non-conductive material, and a secondary non-conductive material arranged to surround a side surface and a lower surface of the non-conductive material.

Holes for fixing and coupling may be formed in preset regions of the polymer material and the non-conductive guide structure, grooves may be formed at locations corresponding to the holes in the non-conductive material and the secondary non-conductive material, and a first screw may be coupled to a first groove formed in the non-conductive material through a first hole formed in an upper portion of the non-conductive guide structure, and a second screw may be coupled to a second groove of the secondary non-conductive material through a second hole formed in a lower portion of the non-conductive guide structure.

Holes for fixing and coupling may be formed in predetermined regions of the polymer material, the non-conductive guide structure, the non-conductive material, and the secondary non-conductive material, and a third screw may pass through the holes, may pass through a lower portion of the non-conductive guide structure, and may be coupled to a nut.

The polymer material may be disposed on a metal material, the polymer material may be bonded to the non-conductive material connected to both end regions of the metal material, the non-conductive guide structure may be provided in a plate shape, holes for fixing and coupling may be formed in the polymer material, the non-conductive guide structure, the non-conductive material, and a secondary non-conductive material disposed to surround a side surface and a lower surface of the non-conductive material, and a fourth screw may pass through the holes, may pass through a lower portion of the non-conductive guide structure, and may be coupled to a nut.

The polymer material may be disposed on a metal material, the polymer material may be bonded to the non-conductive material connected to both end regions of the metal material, the non-conductive guide structure may be provided in a “I” shape, holes for fixing and coupling may be formed in the polymer material, the non-conductive guide structure, the non-conductive material, and a secondary non-conductive material arranged to surround a side surface and a lower surface of the non-conductive material, and a fifth screw may pass through the holes, may pass through a lower portion of the secondary non-conductive material, and may be coupled to a nut.

A medical ultrasonic triboelectric generator structure for charging a body implantable device according to the present disclosure includes a metal material, a polymer material disposed on the metal material, a non-conductive material disposed to surround a side surface and a lower surface of the metal material, a secondary non-conductive material disposed to surround a side surface and a lower surface of the non-conductive material, and a physical guide structure including a non-conductive guide structure and a fixing and coupling structure disposed to reinforce adhesion between the metal material and the non-conductive material.

The non-conductive guide structure may be provided in a “c” shape and may be disposed to surround partial regions of the polymer material, the non-conductive material, and the secondary non-conductive material.

Holes may be formed in preset regions of the polymer material and the non-conductive guide structure, grooves may be formed at locations corresponding to the holes in the non-conductive material and the secondary non-conductive material, and the fixing and coupling structure may include a first screw coupled to a first groove formed in the non-conductive material through a first hole formed in an upper portion of the non-conductive guide structure and a second screw coupled to a second groove formed in the secondary non-conductive material through a second hole formed in a lower portion of the non-conductive guide structure.

The fixing and coupling structure may include a third screw passing through holes formed in the polymer material, the non-conductive guide structure, the non-conductive material, and the secondary non-conductive material and a nut coupled to the third screw.

The non-conductive guide structure may be provided in a “I” shape and may be disposed over upper surfaces of the non-conductive material and the secondary non-conductive material and a partial side region of the secondary non-conductive material.

The fixing and coupling structure may include a fifth screw passing through holes formed in the polymer material, the non-conductive guide structure, the non-conductive material, and the secondary non-conductive material and a nut coupled to the fifth screw.

The medical ultrasonic triboelectric generator structure for charging a body implantable device according to the present disclosure may further include a non-conductive guide disposed between the polymer material and the metal material.

The medical ultrasonic triboelectric generator structure may further include a non-adsorbent disposed on an upper surface of the metal material, and a height of the non-adsorbent may be relatively lower than a height of the non-conductive guide with respect to the upper surface of the metal material.

The non-adsorbent may be made of copper or gold and have a horizontal cross section formed in a quadrangular shape or a circular shape.

An area of the non-adsorbent may be 1/10 of an area of a horizontal cross-sectional area of the medical ultrasonic triboelectric generator structure for charging a body implantable device.

The plurality of non-adsorbents may be disposed and a total area of the plurality of non-adsorbents may be 1/10 of an area of the horizontal cross-sectional area of the medical ultrasonic triboelectric generator structure for charging a body implantable device.

### BRIEF DESCRIPTION OF DRAWINGS

The above and other objects, features and advantages of the present disclosure will become more apparent to those of ordinary skill in the art by describing exemplary embodiments thereof in detail with reference to the accompanying drawings, in which:

FIG. 1 illustrates a configuration of a triboelectric charging generator according to a related art;

FIG. 2A illustrates a side surface of a triboelectric charging generator structure according to the related art, and FIG. 2B illustrates an upper part of the triboelectric charging generator structure according to the related art;

FIGS. 3A to 3D illustrate a side surface of an adhesion improvement structure using a physical guide structure and a fixing screw according to an embodiment of the present disclosure, and FIG. 3E illustrates an upper portion of the structure according to the embodiment of the present disclosure;

FIGS. 4A and 4B illustrate a side surface of a triboelectric generator (TENG) configured in multiple layers using the physical guide structure and the fixing screw according to an embodiment of the present disclosure;

FIG. 5 illustrates a concept of protecting an adhesive part in a high-speed vibration energy source such as ultrasonic waves according to an embodiment of the present disclosure; and

FIG. 6 illustrates a method for forming a medial ultrasonic triboelectric generator structure for charging a body implantable device according to an embodiment of the present disclosure.

### DETAILED DESCRIPTION

The above-described purpose, other purposes, advantages, and features of the present disclosure and a method of achieving the above-described purpose, other purposes, advantages, and features will become apparent with reference to embodiments described below in detail together with the accompanying drawings.

However, the present disclosure is not limited to the embodiments disclosed below and may be implemented in various different forms, the following embodiments are merely provided to easily inform those skilled in the art to which the present disclosure pertains of the purpose, the configuration, and the effect of the present disclosure, and the scope of the present disclosure is defined by the appended claims.

Meanwhile, terms used in the present specification are intended to describe the embodiments and are not intended to limit the present disclosure. In the present specification, a singular form also includes a plural form unless specifically mentioned in a phrase. The term “comprise” and/or “comprising” used herein means that components, steps, operations, and/or elements described above do not exclude the presence or addition of one or more other components, steps, operations, and/or elements.

Polymer materials such as polytetrafluoroethylene (PTFE), perfluoroalkoxy alkanes (PFA), and polyvinylidene fluoride (PVDF), which are mainly used in a triboelectric charging generator, are bonded to other materials.

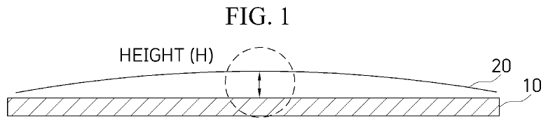

Referring to FIG. 1, a metal material 10 and a polymer material 20 are utilized, and in this case, an output voltage is high, but there is a bonding issue between two materials.

Referring to FIG. 1, two different materials are not bonded to each other (that is, surfaces of two objects are not in contact with and separated from each other), a displacement h between two materials rubbing against each other due to vibrations is present, and energy is generated due to the rubbing.

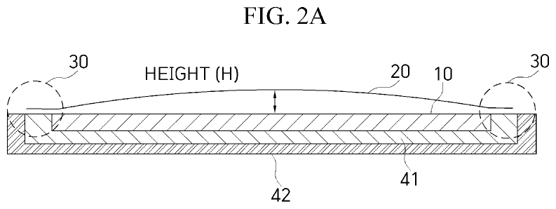

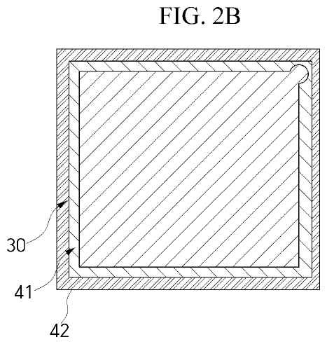

This triboelectric charging method is defined as a triboelectric generator (TENG). FIG. 2A illustrates a side surface of an element structure, and FIG. 2B illustrates an upper part of the element structure.

That is, the metal material 10 and the polymer material 20 are arranged with a space having the height h interposed therebetween, a non-conductive material 41 and a secondary non-conductive material 42 are dually arranged, and thus as much triboelectrically charged energy is transmitted as possible.

In this case, bonding of the polymer material 20 and the non-conductive material 41 is required in an adhesion part 30, and in general, when a contact surface is 50×50 mm² or less, the contact surface does not exceed a specific adhesion area (6%) to optimize output.

That is, the area of the adhesion part 30 should be minimized, and PTFE or PFA used as the polymer material 20 is composed of a diflluoromethalyne (CF2) chain and a typical fluoropolymer. The fluoropolymer has various advantages in terms of excellent water repellency, high chemical resistance, a weatherproofing property, and an excellent sliding property, but has problems that the surface energy is low and the fluoropolymer is not easily bonded to the other types of materials.

To solve these problems, a chemical etching method has been proposed, but is not preferred because the chemical etching method is harmful to an environment and causes coloring.

Further, a bonding technique and an oxygen plasma processing method through a heat-based plasma process have been proposed. However, these bonding methods may be useful at a low power generation frequency of 10 Hz or less but mostly have a low adhesive strength of 1 N/mm to 2.5 N/mm. Thus, when an element vibrates at a high speed by an external source, a short circuit occurs, and foreign substances are introduced from the outside.

That is, it is difficult to utilize these bonding methods in a power generation industry in which ultra-high reliability should be ensured in an environment which requires a power generation frequency of several hundred Hz, several hundred kHz, or several MHz or in environment which requires the power generation frequency to be low.

The present disclosure has been proposed to solve the above problems and proposes a physical guide structure for maintaining adhesion and preventing inflow of external foreign substances after an O₂-based plasma process. According to the present disclosure, it is possible to prevent a short circuit between a polymer material and a metal material, reduce noise, and ensure reliability.

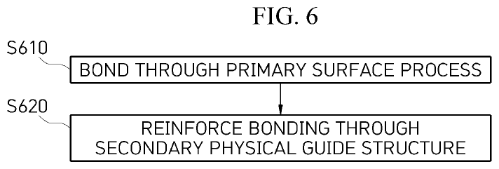

A bonding method for improving power generation of a power generator using a surface process and the physical guide structure according to an embodiment of the present disclosure includes an operation S610 (see FIG. 6) of primarily performing bonding through the surface process and an operation S620 (see FIG. 6) of secondarily reinforcing the bonding using the physical guide structure.

In a description of the operation of primarily performing the bonding through the surface process, when the O2-based plasma process is performed to bond the PFA or PTFE used for the TENG to another material, a contact surface of (O—C═O, C═O, C—C) on a surface of the PFA or PTFE increases. The bonding may be achieved using a non-toxic medical polydimethylsiloxane (PDMS)-based UV adhesive.

By primarily performing the plasma (hydroxyapatite) process and the O₂ surface process, the adhesive strength of the surface of the PFA or PTFE is increased to 2.6 N/mm.

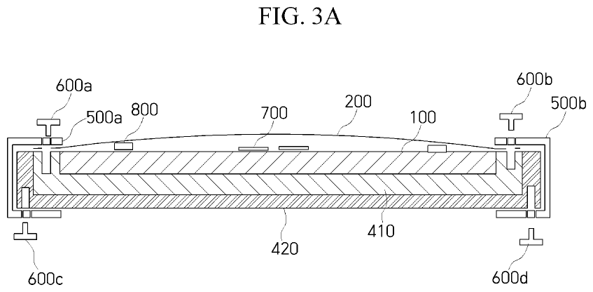

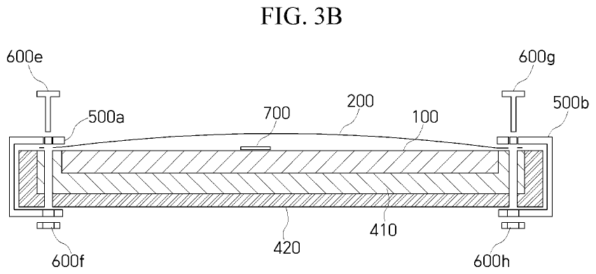

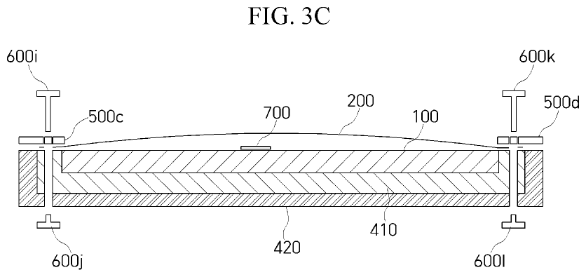

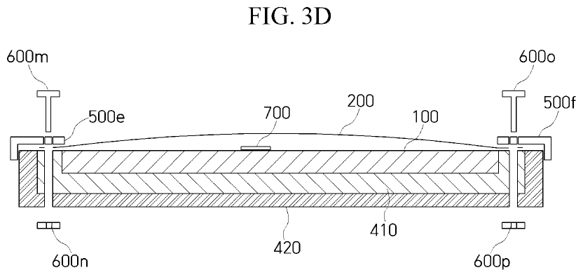

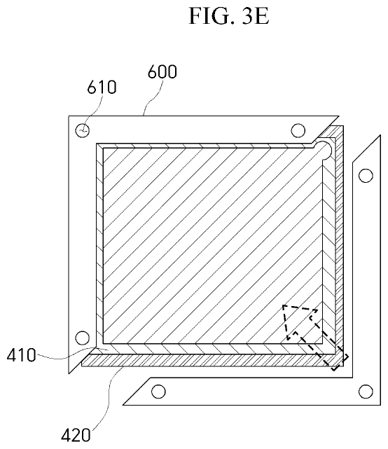

A polymer material 200 (see FIG. 3) and a non-conductive material 410 (see FIG. 3) are bonded to each other using various medical polydimethylsiloxane (PDMS)-based UV adhesives.

In a high-frequency power generation environment, the operation S620 of secondarily reinforcing the bonding using the physical guide structure according to the embodiment of the present disclosure is performed, the bonding is reinforced using the physical guide structure, and thus external shock and noise are reduced, and energy generation is improved.

FIGS. 3A to 3D illustrate a side surface of an adhesion improvement structure using a physical guide structure and a fixing screw according to an embodiment of the present disclosure, and FIG. 3E illustrates an upper portion of the structure according to the embodiment of the present disclosure.

The polymer material 200 is arranged on a metal material 100, and the polymer material 200 is bonded to the non-conductive material 410 in regions of both ends of the metal material 100.

For performing the secondary structural reinforcement, non-conductive guide structures 500a, 500b, 500c, 500d, 500e, and 500f are arranged in adhesion portions.

Adhesion between the non-conductive guide structures 500a, 500b, 500c, 500d, 500e, and 500f, the non-conductive material 410, and a secondary non-conductive material 420 are reinforced using physical pressure caused by fixing coupling structures (illustrated as 600a to 600p and corresponding to screws or nuts).

Referring to FIG. 3A, a non-adsorbent 700 is disposed and a non-conductive guide 800 is disposed in a space between the polymer material 200 and the metal material 100.

For understanding of those skilled in the art, the non-conductive guide 800 is illustrated in FIG. 3A, and the non-conductive guide 800 may also be applied to structures illustrated in FIGS. 3B to 3D.

In the space between the polymer material 200 and the metal material 100, the non-conductive guide 800 is disposed in a region spaced a predetermined distance from a center and the non-adsorbent 700 is disposed in a predetermined region at the center.

The non-adsorbent 700 may be composed of a charged body (CU, AU, or the like) and may also be composed of a non-conductor.

The non-adsorbent 700 is formed of a material such as copper, gold, nickel, aluminum, PVDF, PFA, PTFE, or fluorinated ethylene-propylene (FEP).

The non-adsorbent can be made of metals (copper or gold) or polymers that are same with polymer material or have similar triboelectric series with polymer material for power generation.

As in the side cross-sectional view of FIG. 3A, the height of the non-adsorbent 700 is relatively smaller than the height of the non-conductive guide 800.

When the area of the entire element is 10×10 mm², the non-adsorbent 700 is disposed with an area of lx1 mm².

When an area of the entire element is 20×20 mm², two non-adsorbents 700 having an area of 1×1 mm² are disposed or one non-adsorbent 700 having an area of 1×1 mm² is disposed. In consideration of performance of the amount of power generation, the two non-adsorbents 700 having an area of 1×1 mm² may be disposed.

The non-adsorbent 700 may have any of various shapes such as a quadrangle, a circle, a rhombus, and a pentagon and may be the quadrangle or the circle.

The total area of the non-adsorbent 700 compared to the total area of the power generator is 0.5% or more and 5% or less, and a problem that a positive electrode (a charger) and a negative electrode are bonded to each other can be solved even at a high frequency while reducing loss in the amount of power generation.

In this case, the non-conductive guide structures 500a, 500b, 500c, 500d, 500e, and 500f and the fixing coupling structures 600a to 600p are each formed of a material such as non-conductive plastic (ABS).

According to the embodiment of the present disclosure, the adhesive strength is increased to 3.3 N/cm to 5.5 N/cm, and thus even when the TENG is used in a MHz band environment, high power generation efficiency is ensured.

Referring to FIG. 3A, the non-conductive guide structures 500a and 500b are provided in a “c” shape and are arranged in a shape surrounding both ends of the polymer material 200, the non-conductive material 410, and the secondary non-conductive material 420.

Grooves or holes for coupling the fixing coupling structures are formed in the polymer material 200, the non-conductive material 410, and the secondary non-conductive material 420.

Referring to FIG. 3A, the screws 600a and 600b are coupled to the grooves of the non-conductive material 410 through holes formed in upper portions of the non-conductive guide structures 500a and 500b, and the screws 600c and 600d are coupled to the grooves of the secondary non-conductive material 420 through holes formed in lower portions of the non-conductive guide structures 500a and 500b.

Referring to FIG. 3B, the non-conductive guide structures 500a and 500b are provided in a “c” shape and are arranged in a shape surrounding both ends of the polymer material 200, the non-conductive material 410, and the secondary non-conductive material 420.

Holes for coupling the fixing coupling structures are formed in the polymer material 200, the non-conductive material 410, and the secondary non-conductive material 420.

Referring to FIG. 3B, the screws 600e and 600g pass through the holes formed in upper and lower portions of the non-conductive guide structures 500a and 500b and the holes formed at corresponding locations of the non-conductive material 410 and the secondary non-conductive material 420, and are coupled to the nuts 600f and 600h on an opposite side.

Referring to FIG. 3C, the non-conductive guide structures 500c and 500d are provided in a plate shape and are disposed at both upper end regions of the polymer material 200, the non-conductive material 410, and the secondary non-conductive material 420.

Holes for coupling the fixing coupling structures are formed in the polymer material 200, the non-conductive material 410, and the secondary non-conductive material 420.

Referring to FIG. 3C, the screws 600i and 600k pass through holes formed in the non-conductive guide structures 500c and 500d and the holes formed at corresponding locations of the non-conductive material 410 and the secondary non-conductive material 420, and are coupled to the nuts 600j and 600l on an opposite side.

Referring to FIG. 3D, the non-conductive guide structures 500e and 500f are provided in a “I” shape and are arranged in a shape surrounding both upper end regions of the polymer material 200, the non-conductive material 410, and the secondary non-conductive material 420.

Holes for coupling the fixing coupling structures are formed in the polymer material 200, the non-conductive material 410, and the secondary non-conductive material 420.

Referring to FIG. 3D, the screws 600m and 600o pass through holes formed in the non-conductive guide structures 500e and 500f and the holes formed at corresponding locations of the non-conductive material 410 and the secondary non-conductive material 420, and are coupled to the nuts 600n and 600p on an opposite side.

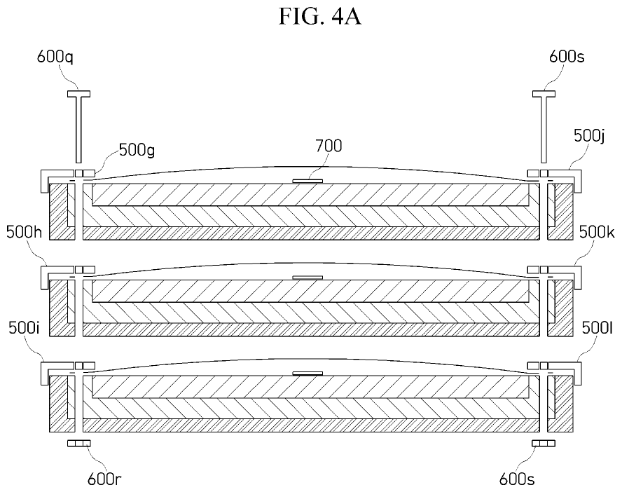

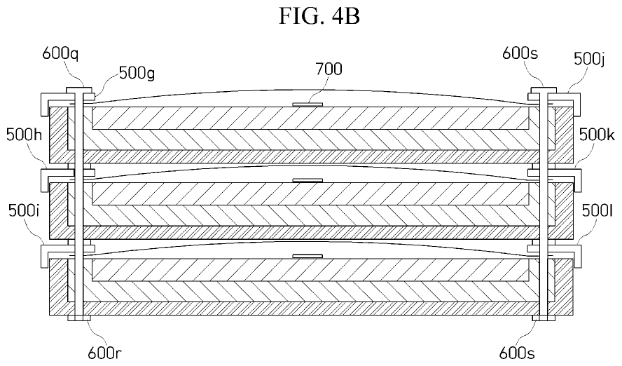

FIGS. 4A and 4B illustrate a side surface of a TENG configured in multiple layers using the physical guide structure and the fixing screw according to an embodiment of the present disclosure.

The non-conductive guide structures 500g, 500h, 500i, 500j, 500k, and 500l are provided in a “I” shape to surround both upper end regions of the polymer material, the non-conductive material, and the secondary non-conductive material which are configured in multiple layers.

Holes for coupling the fixing coupling structures are formed in the polymer material, the non-conductive material, and the secondary non-conductive material which are configured in multiple layers.

Referring to FIGS. 4A and 4B, the screws 600q and 600s pass through holes formed in upper portions of the non-conductive guide structures 500g, 500h, and 500i, and 500j, 500k, and 500l, respectively, and the holes formed at corresponding locations of the non-conductive material and the secondary non-conductive material, and are coupled to the nuts 600r and 600s on an opposite side.

When the TENG is configured in multiple layers, the complexity of a process is high in order to perform the bonding of the layers while maintaining a certain distance between the layers.

According to the embodiment of the present disclosure, since close contact between the layers is performed through the non-conductive guide structure and the fixing screw at regular intervals, high output can be ensured even in the power generator configured as a plurality of (for example, three to four) layers.

Further, the bonding of the layers can be achieved through the fixing screw without using a separate adhesive material, and the plurality of layers can be configured.

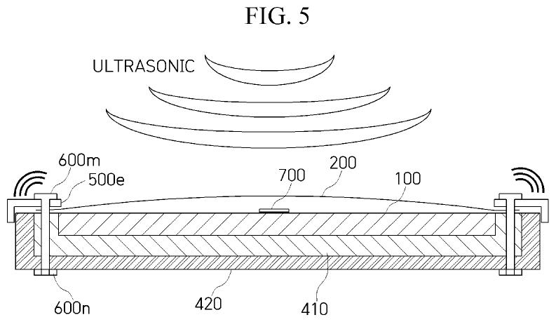

FIG. 5 illustrates a concept of protecting an adhesive part in a high-speed vibration energy source such as ultrasonic waves according to an embodiment of the present disclosure.

Referring to FIG. 5, even when external energy for power generation is ultrasonic waves, the adhesive portion is protected through the non-conductive guide structure, and thus the robustness can be increased. By preventing a short circuit and inflow of foreign substances even in a high-speed vibration energy source, the reliability and safety can be ensured.

Further, by utilizing the physical guide structure in the TENG multi-layer, a defect rate of the bonding of the layers can be reduced, and high assembly convenience as in Lego blocks can be ensured.

Meanwhile, a method of forming a medical ultrasonic triboelectric generator structure for charging a body implantable device according to the embodiment of the present disclosure can be applied to small electronic products, medical devices, and industrial devices.

The ultrasonic triboelectric generator structure according to the embodiment of the present disclosure can generate energy due to friction, and the generated energy can be utilized in a battery or device through a rectifier.

A call bell is exemplified as the small electronic products, and when the power generator is mounted on the call bell, energy is generated by the power generator through a force for pressing the call bell. The generated energy supplies power through a storage or wireless radio frequency (RF) and provides device driving energy to provide a calling function without a separate battery.

Further, in the medical device field, the ultrasonic triboelectric generator structure is applied to a micro sensor or a medical device for in-vivo, generates energy by vibrations while inserted into a body, and the generated energy may be used as a neural stimulation or a power source for the medical device.

The medical device inserted into the body includes a processor, a memory, a biometric sensor, and a wired/wireless output device, and each component performs data communication through an RF module for data communication.

In the industrial device, a sensor having a separate power source is used to measure a vibration state. However, according to the embodiment of the present disclosure, the power generator is vibrated through the kinetic energy generated by the device without a separate additional sensor, and thus the vibration state can be precisely measured using vibration state, direction, and bending information.

The method of forming a medical ultrasonic triboelectric generator structure for charging a body implantable device according to the embodiment of the present disclosure may be implemented in a computer system or recorded on a recording medium. The computer system may include at least one processor, a memory, a user input device, a data communication bus, a user output device, and a storage. The above-described components perform data communication through the data communication bus.

The computer system may further include a network interface coupled to a network. The processor may be a central processing unit (CPU) or a semiconductor device that executes a command stored in the memory and/or the storage.

The memory and storage may include various types of volatile or non-volatile storage media. For example, the memory may include a read-only memory (ROM) and a random-access memory (RAM).

Thus, the method of forming a medical ultrasonic triboelectric generator structure for charging a body implantable device according to the embodiment of the present disclosure may be implemented in a computer-executable manner. When the method of forming a medical ultrasonic triboelectric generator structure for charging a body implantable device according to the embodiment of the present disclosure is performed in a computer device, computer-readable commands may perform the method of forming a medical ultrasonic triboelectric generator structure for charging a body implantable device according to the present disclosure.

Meanwhile, the above-described method of forming a medical ultrasonic triboelectric generator structure for charging a body implantable device according to the embodiment of the present disclosure may be implemented as computer-readable code on a computer-readable recording medium. The computer-readable recording medium includes any type of recording medium in which data that may be read by the computer system is stored. Examples of the computer-readable recording medium include a ROM, a RAM, a magnetic tape, a magnetic disk, a flash memory, an optical data storage device, and the like. For example, computer-readable recording media may be distributed in the computer system connected through a computer communication network and stored and executed as readable code in a distributed manner.

According to the present disclosure, a plasma surface process is performed to bond an insulator (PFA or PTFE) used in a configuration of the TENG and another material, adhesive fixation is performed using a physical guide structure for high frequency power generation, and thus external shock is reduced, power generation noise is reduced, and energy generation efficiency can be increased.

Further, when the TENG is configured in multiple layers, the layers come into close contact with each other using the physical guide structure and a fixing screw, a distance (see h in FIG. 1) between a metal material and a polymer material is maintained at a constant value, and thus high output is ensured.

Further, even when a high-speed vibration energy source such as ultrasonic waves is used, a non-conductive physical guide structure is used to prevent a short circuit and inflow of foreign substances and ensure reliability and safety.

The effects of the present disclosure are not limited to the effects described above, and other effects not described will be clearly understood by those skilled in the art from the above detailed description.

What is claimed is:

1. A method of forming a medical ultrasonic triboelectric generator structure for charging a body implantable device, the method comprising:
(a) primarily performing a plasma process on a power generation material on which a polymer material is disposed and performing bonding of the polymer material and a non-conductive material; and
(b) secondarily reinforcing the bonding of the polymer material and the non-conductive material using a physical guide structure including a non-conductive guide structure and a fixing coupling structure.

2. The method of claim 1, wherein the polymer material is disposed on a metal material and bonded to the non-conductive material in preset regions at both ends of the metal material, and the non-conductive guide structure is provided in a “⊏” shape and is disposed in a shape surrounding partial regions of the polymer material, the non-conductive material, and a secondary non-conductive material arranged to surround a side surface and a lower surface of the non-conductive material.

3. The method of claim 2, wherein holes for fixing and coupling are formed in preset regions of the polymer material and the non-conductive guide structure,
grooves are formed at locations corresponding to the holes in the non-conductive material and the secondary non-conductive material, and
a first screw is coupled to a first groove formed in the non-conductive material through a first hole formed in an upper portion of the non-conductive guide structure, and a second screw is coupled to a second groove of the secondary non-conductive material through a second hole formed in a lower portion of the non-conductive guide structure.

4. The method of claim 2, wherein holes for fixing and coupling are formed in predetermined regions of the polymer material, the non-conductive guide structure, the non-conductive material, and the secondary non-conductive material, and a third screw passes through the holes, passes through a lower portion of the non-conductive guide structure, and is coupled to a nut.

5. The method of claim 1, wherein the polymer material is disposed on a metal material,
the polymer material is bonded to the non-conductive material connected to both end regions of the metal material,
the non-conductive guide structure is provided in a plate shape,
holes for fixing and coupling are formed in the polymer material, the non-conductive guide structure, the non-conductive material, and a secondary non-conductive material,
the secondary non-conductive material is disposed to surround a side surface and a lower surface of the non-conductive material, and
a fourth screw passes through the holes, passes through a lower portion of the non-conductive guide structure, and is coupled to a nut.

6. The method of claim 1, wherein the polymer material is disposed on a metal material,
the polymer material is bonded to the non-conductive material connected to both end regions of the metal material,
the non-conductive guide structure is provided in a “I” shape,
holes for fixing and coupling are formed in the polymer material, the non-conductive guide structure, the non-conductive material, and a secondary non-conductive material,
the secondary non-conductive material is disposed to surround a side surface and a lower surface of the non-conductive material, and
a fifth screw passes through the holes, passes through a lower portion of the secondary non-conductive material, and is coupled to a nut.

7. A medical ultrasonic triboelectric generator structure for charging a body implantable device, the structure comprising:
a metal material;
a polymer material disposed on the metal material;
a non-conductive material disposed to surround a side surface and a lower surface of the metal material;
a secondary non-conductive material disposed to surround a side surface and a lower surface of the non-conductive material; and
a physical guide structure including a non-conductive guide structure and a fixing and coupling structure disposed to reinforce adhesion between the metal material and the non-conductive material.

8. The medical ultrasonic triboelectric generator structure of claim 7, wherein the non-conductive guide structure is provided in a “c” shape and is disposed to surround partial regions of the polymer material, the non-conductive material, and the secondary non-conductive material.

9. The medical ultrasonic triboelectric generator structure of claim 8, wherein holes are formed in preset regions of the polymer material and the non-conductive guide structure,
grooves are formed at locations corresponding to the holes in the non-conductive material and the secondary non-conductive material, and
the fixing and coupling structure include a first screw coupled to a first groove formed in the non-conductive material through a first hole formed in an upper portion of the non-conductive guide structure and a second screw coupled to a second groove formed in the secondary non-conductive material through a second hole formed in a lower portion of the non-conductive guide structure.

10. The medical ultrasonic triboelectric generator structure of claim 8, wherein the fixing and coupling structure includes a third screw passing through holes formed in the polymer material, the non-conductive guide structure, the non-conductive material, and the secondary non-conductive material and a nut coupled to the third screw.

11. The medical ultrasonic triboelectric generator structure of claim 7, wherein the non-conductive guide structure is provided in a “┐” shape and is disposed over upper surfaces of the non-conductive material and the secondary non-conductive material and a partial side region of the secondary non-conductive material.

12. The medical ultrasonic triboelectric generator structure of claim 11, wherein the fixing and coupling structure includes a fifth screw passing through holes formed in the polymer material, the non-conductive guide structure, the non-conductive material, and the secondary non-conductive material and a nut coupled to the fifth screw.

13. The medical ultrasonic triboelectric generator structure of claim 7, further comprising a non-conductive guide disposed between the polymer material and the metal material.

14. The medical ultrasonic triboelectric generator structure of claim 13, further comprising a non-adsorbent disposed on an upper surface of the metal material,
wherein a height of the non-adsorbent is relatively lower than a height of the non-conductive guide with respect to the upper surface of the metal material.

15. The medical ultrasonic triboelectric generator structure of claim 14, wherein the non-adsorbent has a horizontal cross section formed in a quadrangular shape or a circular shape.

16. The medical ultrasonic triboelectric generator structure of claim 14, wherein an area of the non-adsorbent is 1/10 of an area of a horizontal cross-sectional area of the medical ultrasonic triboelectric generator structure for charging a body implantable device.

17. The medical ultrasonic triboelectric generator structure of claim 16, wherein the plurality of non-adsorbents are disposed and a total area of the plurality of non-adsorbents is 1/10 of an area of the horizontal cross-sectional area of the medical ultrasonic triboelectric generator structure for charging a body implantable device.
